(19) United States
(12) Patent Application Publication
(10) Pub. No.: US 2010/0217265 A1
(43) Pub. Date: Aug. 26, 2010

(54) ANTI-SUBSIDENCE DYNAMIC HIP SCREW

(75) Inventors: Tain-Hsiung CHEN, Taipei City (TW); Cheng-Kung CHENG, Taipei City (TW); Hung-Wen WEI, Taipei City (TW); Yu-Shu LAI, Taipei City (TW); Ping-Sheng YU, Taipei City (TW)

Correspondence Address: Muncy, Geissler, Olds & Lowe, PLLC, 4000 Legato Road, Suite 310, FAIRFAX, VA, 22033, US

(73) Assignees: NATIONAL YANG-MING UNIVERSITY, Taipei City (TW); TAIPEI VETERANS GENERAL HOSPITAL, Taipei (TW)

(21) Appl. No.: 12/470,899

(22) Filed: May 22, 2009

(30) Foreign Application Priority Data
Feb. 24, 2009 (TW) 098105867

(51) Int. Cl. A61B 17/56

(52) U.S. Cl. 606/64; 606/65

(57) ABSTRACT

An anti-subsidence dynamic hip screw comprises of a barrel having a connection portion and a tube-type extending portion angled a predetermined angle with the connection portion, and a first long groove and a first limit portion disposed at the inner wall of the extending portion respectively; a lag screw having an inside end disposed threading screw at its outer edge and a outside end with a predetermined length passed through in the extending portion by the way of reciprocation; a second long groove and a second limit portion disposed at the lag screw respectively related to the first long groove and the first limit portion, each first limit portion and second limit portion used for preventing the lag screw from rotating inside the extending portion; a blade piece with a predetermined length simultaneously passed through and retained in the first long groove and the related second long groove; a compressing screw having an inside end screwed with the outside end of the lag screw and an outside end retained in the outside end of the extending portion. The above structure is not only easy to assembly, especially for the effect of decreasing the subsidence of the femoral head, but also makes the effect more obvious when using for the hip fracture of the osteoporosis body.

### BACKGROUND OF THE INVENTION

1. Field of the Invention

The present invention generally relates to an anti-subsidence dynamic hip screw, and more particularly to an anti-subsidence dynamic hip screw capable of decreasing the subsidence of the femoral head when using for reconstructing and resetting femoral fractures.

2. Description of the Related Art

Clinically, the proximal femoral fracture is one of the common diseases and occurred in the eldly and the population of the osteoporosis. Because the osteoporosis results in the fractures of the femoral neck or greater trochanter of the hip joint, it is usually used the dynamic hip screw or compression hip screw to reconstruct and reset the fracture.

The design of conventional compression hip screw emphasizes on enhancing the lock of the screw (disclosed by U.S. Pat. No. 4,973,333 and U.S. Pat. No. 5,041,116), preventing the rotation of the femoral head (disclosed by U.S. Pat. No. 4,657,001), or providing the better fixing way of the screw (disclosed by U.S. Pat. No. 6,511,481 and U.S. Pat. No. 7,118,572).

However, when the screws of above patents disclosed is used in the osteoporosis patients, the bone mass of the femoral head is too low and results in the femoral head collapsing excessively so that the contact area between the lag screw locked in the femoral head and the bone is too small and further results in the complications of superior cut-out of the lag screw

Although U.S. Pat. No. 7,118,572 provides a lag screw assembly with better fixing function which the lag screw is disposed four tang legs and spreads out after the lag screw anchors into the femoral head so as to enhance the fixing effect. However, the contact area between the tang legs and the femur is not large enough, and then it is not obvious to fix the lag screw to the femoral head when encountering the situation of above mentioned that the femoral head collapses excessively because the bone mass is too low.

The U.S. Pat. No. 4,657,001 provides an anti-rotational hip screw capable of increasing the contact area with the femur, which achieves the objective of increasing contact area by the four aligned grooves disposed at the edges of the screw and four pins implanted into the femoral neck. However, the depth of each pin implanting into the femur is limited. Therefore, the effect of the subsidence relative to the femoral head is limited, too.

### SUMMARY OF THE INVENTION

An anti-subsidence dynamic hip screw of the present invention is comprising: a barrel having a connection portion and an extending portion connecting with an upper end of the connection portion by an outside end of the extending portion, and there is a predetermined angle between the connection portion and the extending portion so as to become a bending form, at least one first long groove disposed at a predetermined position of the extending portion and axially extended between an inside end and an outside end of the first groove, a stop portion disposed at the predetermined position; a lag screw having an inside end with threading screw at the outer edge of the inside end and an outside end with a predetermined length to combine with the extending portion by the way of reciprocation, at least one second long groove disposed at an outer edge of the lag screw and axially extending a predetermined length; a blade piece having a predetermined length and a predetermined width and simultaneously passed through the first long groove and the second long groove corresponding to each other; and a compressing screw having an inside end screwed with the outside of the lag screw, and an outside end limited by the stop portion to retain in the outside end of the extending portion.

According to above structure, it is not only easy to assembly, especially for the effect of decreasing the subsidence of the femoral head, but also makes the effect more obvious when using for the hip fracture of the osteoporosis body.

Further features and advantages of the present invention will become apparent to those of skill in the art in view of the detailed description of preferred embodiments which follows, when considered together with the attached drawings and claims.

### BRIEF DESCRIPTION OF THE DRAWINGS

All the objects, advantages, and novel features of the invention will become more apparent from the following detailed descriptions when taken in conjunction with the accompanying drawings.

FIG. 1 shows a perspective drawing of a preferred embodiment in accordance with the invention;

FIG. 2 shows an exploded assembly drawing of the preferred embodiment in accordance with the invention;

FIG. 3 shows a sectional drawing of the extending portion of the preferred embodiment in accordance with the invention;

FIG. 4 shows a sectional drawing of the preferred embodiment in accordance with the invention;

FIG. 5 shows an enlarged drawing of the lag screw of the preferred embodiment in accordance with the invention; and

FIG. 6 shows a sectional drawing of the preferred embodiment along the 6-6 direction of FIG. 1.

FIG. 7 shows a perspective schematic drawing when the preferred embodiment is in use.

### DETAILED DESCRIPTION OF THE PREFERRED EMBODIMENT

Referring now to the drawings where like characteristics and features among the various figures are denoted by like reference characters. The present invention is disclosed a dynamic hip combination structure used for reconstructing the femoral head fractures, which mainly comprises of a barrel 1, a lag screw 2, two blade piece 3 and a compressing screw 4.

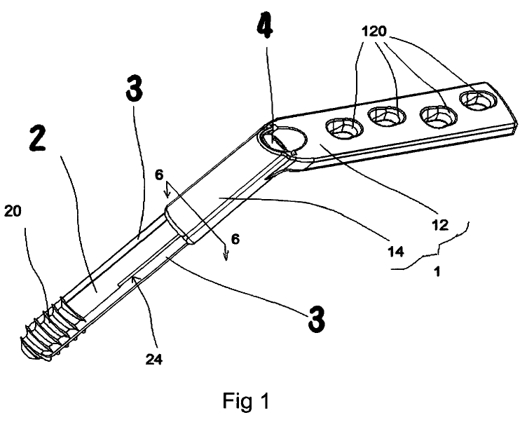

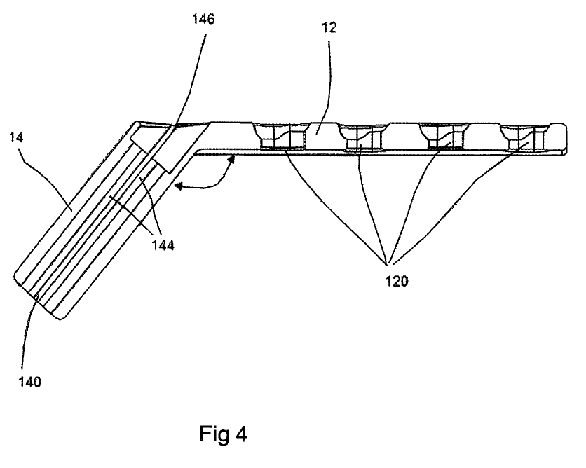

The barrel 1, as shown in FIG. 1 to FIG. 4, has a connection portion 12 which the inside of the connection portion 12 is a concave arc surface for increase the contact area of the connection portion 12 and the outer edge of a femur when setting the connection portion against the outer edge of the femur, and four through holes 120 is passed through the inside and outside of the connection portion 12 and spaced apart an adaptive distance; and an extending portion 14 is tube type with an adaptive length, which the outer edge of the outside end of the extending portion 14 is connected with the upper end of the connection portion 12 and an inner angle is 120˜140 degree axially disposed therebetween so that the barrel 1 becomes a bending form. The inner diameter of a shaft hole 140 inside the extending portion 14 adjacent to the outside end of the connection portion 12 is larger than the one of the inside end, and a stop portion 142 is disposed at a shoulder of the neighbor between the connection portion 12 and the extending portion 14. Two first limit portions 144 are the planes disposed at the inner wall of the shaft hole 140 respectively and extend an adaptive length from the outside end to the inside end so that the sectional plane of the portion relative to the shaft hole 140 forms ellipse shape. Moreover, the two first long grooves 146 are disposed at the inner wall of the shaft hole 140 and extend an adaptive length from the outside end to the inside end. In this embodiment, the two first limit portions are angled 180 degree and the two first long grooves are concaved to each related first limit portion 144.

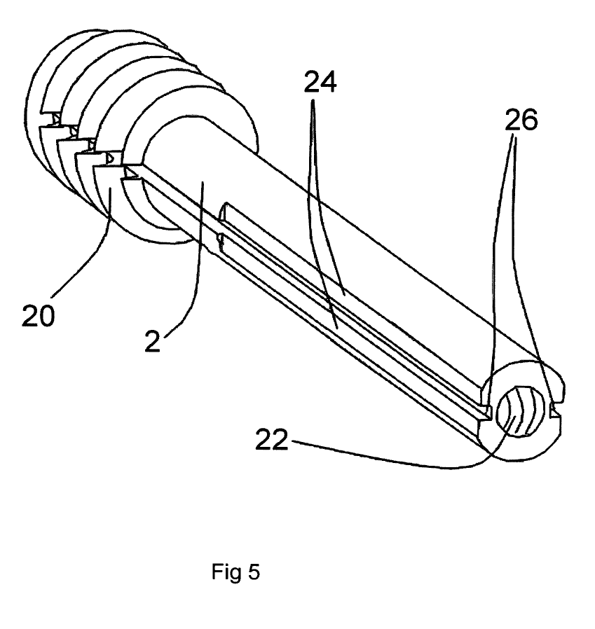

The lag screw 2, as shown in FIG. 5, is hollow type with an adaptive length, which the outside end of the lag screw 2 has an adaptive length to pass through and retain in the extending portion 14 by the way of reciprocation, the outer edge of the inside end of the lag screw 2 has threading screw 20, and the inner wall of the outside end of the lag screw 2 has inner threads 22. Two second limit portions 24 are disposed at the plane of the outer edge of the lag screw 2 respectively and extend an adaptive length from outside end to the inside end. Two second long grooves 26 are concaved on the outer edge and axially extend an adaptive length, and extend an adaptive length from the outside end to the inside end when passing the second limit portions 24. When the extending portion 14 of the barrel 1 is put onto out of the outside end of the lag screw 2, each first limit portion 144 and related second limit portion 24 are set against each other and each first long groove and related second long groove are connected each other.

The two blade pieces 3, as shown in FIGS. 1, 2 and 6, are strip type with an adaptive thickness and an adaptive width and have rectangular cross sections, and are simultaneously wedged in each first long groove 146 and second long groove 26. A lead angle is disposed at the inside ends of the blade pieces 3 so as to reduce the friction or prevent from deviating the axial center in the implanting process. If necessary, the lead angle can be blade type. A through hole 30 is disposed at the outside end of the blades 3 so as to cooperate with other means (not shown) to remove the two blade pieces 3 which have been disposed in the femur.

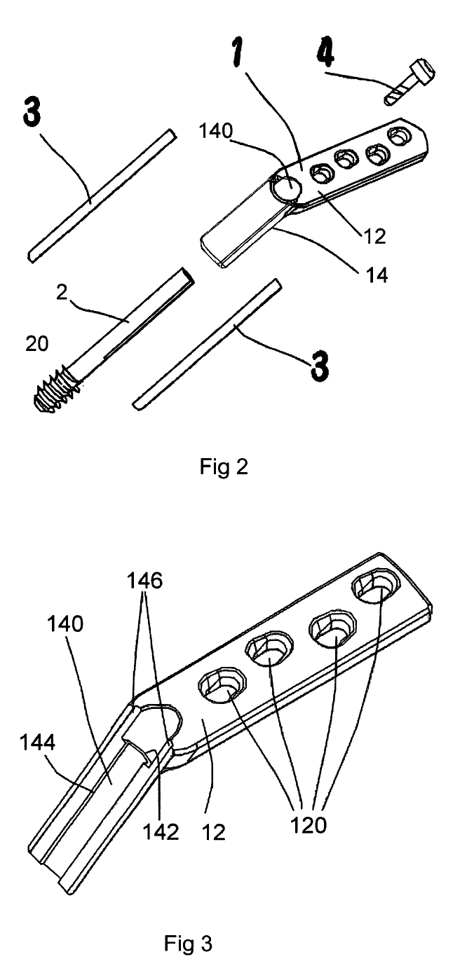

The inside end of the compressing screw 4, as shown in FIGS. 1 and 2, is used for screwing with the inner threads 22 of the outside end of the lag screw 2, the outside end with larger outer diameter is formed an adaptive shape to cooperate with a screwdriver or a hex key wrench, and the rotation of the compressing screw 4 is always retained in the outside end of the shaft hole 140 of the extending portion 14 that is stopped by the stop portion 142 because the outer diameter of the outside end of the compressing screw 4 is larger than the inner diameter of the inside end of the shaft hole 140.

When operating, the lag screw 2 is screwed into the fracture of the femoral head first, the extending portion 14 of the barrel I is put onto the outside end of the lag screw 2 and each first limit portion 144 and second limit portion 24 are disposed relatively each other to prevent the lag screw 2 from rotating therein, and then the connection portion 12 of the barrel 1 is fixed to the femur through the through hole 120 by four fixation bolts (not shown).

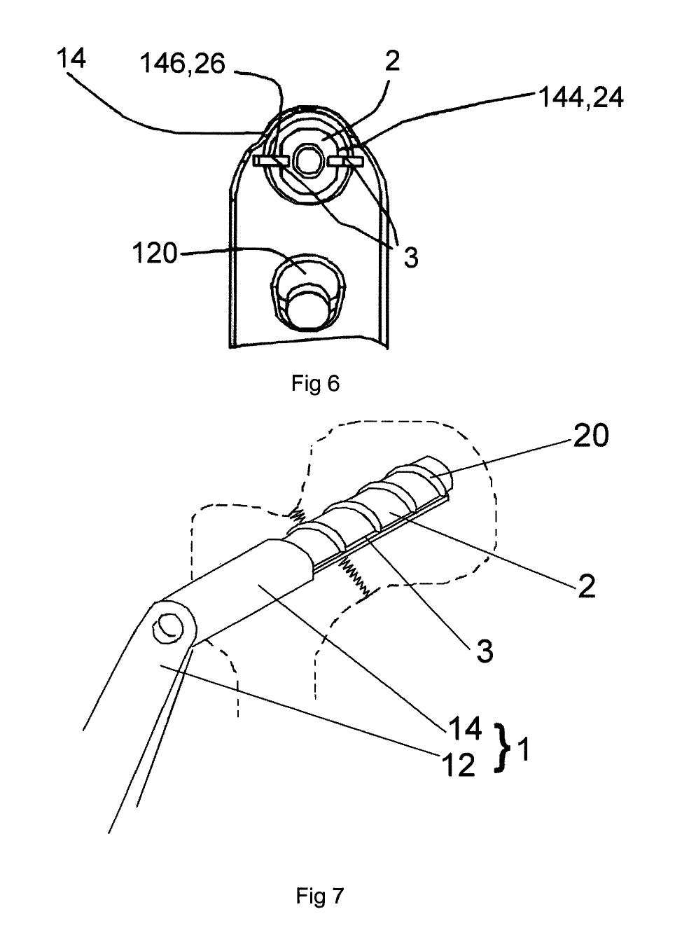

Subsequently, the two blade pieces 3 are wedged in each first long groove 146 and second long groove 26 against each other, as shown in FIG. 6, and the blade pieces 3 should bury in respective long grooves 146, 26 when the length of each blade piece 3 is not larger than the one of each long groove 146, 26 so that the outside ends of the blade pieces 3 are not protruded out of the outside end of the lag screw 2 and the inside ends of the blade pieces 3 have an adaptive length to extend to the inside of the femoral head, as shown in FIG. 7, and the rectangular inside end of each blade piece 3 contacts with the femoral head, so as to increase the contact area therebetween. Additionally, the height of each blade piece 3 protruded from the outer edge of the lag screw 2 is set to be higher than or equal to the height of the threading screw 20, so as to increase the contact area much more than above mentioned.

Finally, rotate the compressing screw 4 after the compressing screw 4 is inserted from the outside end of the extending portion 14 and screwed with the outside end of the lag screw 4. Since the outside end of the compressing screw 4 is stopped by the stop portion 142 of the extending portion 14 and each first limit portion 144 is set against each related second limit portion 24, operator rotates the compressing screw 4 through the barrel 1 and the lag screw 4 to tow two relative fracture position to reset.

In the embodiment, it is effectively increasing the contact area between the cross-lock combination structure and the femoral head by the two wing-type blade pieces 3, as shown in FIGS. 1 an 7, protruded the two sides of the lag screw 4, which is not only enhancing the fixing effect, but also achieving the effect of decreasing the subsidence of the femoral head.

Due to the inside ends of the above mentioned blade pieces 3 are protruded an adaptive length out of the extending portion 14, in practical operation, operator is able to drill a strip hole (not shown) in the femoral head adjacent to the outsides of the two second long grooves 26 so that the insides of the two blade pieces 3 extend to the inside of the femoral head.

In above mentioned embodiment, the quantity and the position of each blade piece 3 is a best implementation. In practical application, the quantity is not only able to adjust considerably, but also the each related long groove 146, 26 is able to be disposed at another non-planar positions excluding each first limit portion 144 or second limit portion 24.

Although the invention has been explained in relation to its preferred embodiment, it is not used to limit the invention. It is to be understood that many other possible modifications and variations can be made by those skilled in the art without departing from the spirit and scope of the invention as hereinafter claimed.

What is claimed is:

1. An anti-subsidence dynamic hip screw, comprising:
a barrel having a connection portion and an extending portion connecting with an upper end of the connection portion by an outside end of the extending portion, and there is a predetermined angle between the connection portion and the extending portion so as to become a bending form, at least one first long groove disposed at a predetermined position of the extending portion and axially extended between an inside end and an outside end of the first groove, a stop portion disposed at the predetermined position;
a lag screw having an inside end with a threading screw at the outer edge of the inside end and an outside end with a predetermined length to combine with the extending portion by the way of reciprocation, at least one second long groove disposed at an outer edge of the lag screw and axially extending a predetermined length;
a blade piece having a predetermined length and a predetermined width and simultaneously passed through the first long groove and the second long groove corresponding to each other, a height of an inside end of the blade piece protruded an outer edge of the grooving bone is not less than a height of the threading screw so as to increase a contact area with the femoral head; and
a compressing screw having an inside end screwed with the outside of the lag screw, and an outside end limited by the stop portion to retain in the outside end of the extending portion and against an outer end of the blade piece not to separate out of the extending portion.

2. The anti-subsidence dynamic hip screw as claimed in claim 1, wherein the inside of the connection portion is a concave arc surface for increase the contact area of the connection portion and the outer edge of the femur when setting the connection portion against the outer edge of a femur.

3. The anti-subsidence dynamic hip screw as claimed in claim 1, wherein the extending portion is tube type and connecting the connection portion with the outer edge of the outside end of the extending portion.

4. The anti-subsidence dynamic hip screw as claimed in claim 3, wherein the extending portion has a shaft hole therein which the inner diameter of the outside end is larger than the one of the inside end, the stop portion is disposed at a shoulder of the neighbor between the connection portion and the extending portion.

5. The anti-subsidence dynamic hip screw as claimed in claim 3, wherein the outside end of the lag screw is passed and disposed in the extending portion.

6. The anti-subsidence dynamic hip screw as claimed in claim 3, wherein the outer diameter of the outside end of the compressing screw is larger than the one of the inside end of the compressing screw and the inner diameter of the inside end of the shaft hole.

7. The anti-subsidence dynamic hip screw as claimed in claim 1, wherein at least one first limit portion is disposed at the predetermined position of the extending portion, at least one second limit portion is disposed at the predetermined position of the lag screw, and when the lag screw combines with the barrel, the first limit portion and the second limit portion set against each other so as to prevent the lag screw from rotating in the operation of reciprocation.

8. The anti-subsidence dynamic hip screw as claimed in claim 7, wherein the extending portion is tube type, the first limit portion is a plane disposed at an inner wall of the extending portion and extending a predetermined length, the outside end of the lag screw is passed and disposed in the extending portion, and the second limit portion is disposed at the plane of the outer edge of the lag screw.

9. The anti-subsidence dynamic hip screw as claimed in claim 7, wherein the first long groove and the second long groove are concaved on the first limit portion and the second limit portion respectively.

10. The anti-subsidence dynamic hip screw as claimed in claim 1, wherein the second long groove is extended from the outside end of the lag screw to the inside end, and the length of the second long groove is less than the one of the lag screw.

11. The anti-subsidence dynamic hip screw as claimed in claim 8, wherein the first long groove and the second long groove are concaved on the first limit portion and the second limit portion respectively.
